(19) United States
(12) Patent Application Publication
(10) Pub. No.: US 2008/0009934 A1
(43) Pub. Date: Jan. 10, 2008

(54) ENDOPROSTHESIS DELIVERY SYSTEM WITH STENT HOLDER

(75) Inventors: Eric Schneider, Albion, RI (US); Mark Wood, Watertown, MA (US); Gary J. Leanna, Holden, MA (US); Gary Jordan, Litchfield, NH (US); James Weldon, Newton, MA (US); Jason Weiner, Brighton, MA (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC., Maple Grove, MN (US)

(21) Appl. No.: 11/762,334

(22) Filed: Jun. 13, 2007

Related U.S. Application Data
(60) Provisional application No. 60/819,422, filed on Jul. 7, 2006.

(51) Int. Cl. A61F 2/06

(52) U.S. Cl. 623/1.11; 623/1.15

(57) ABSTRACT

A device, such as a catheter, for intraluminally delivering a distensible stent includes a first elongate tubular member having a proximal end and a distal end; a band circumferentially disposed over at least a circumferential portion of the first tubular member at the proximal end, the band having at least one projection for releasably engaging a portion of a radially distensible stent; and a second elongate tubular member slidably disposed over the first tubular member and the band. Desirably, the at least one projection is a low-profile, lobate-projection. shaped projection. The band may include two opposed projections, where the two opposed projections may be circumferentially disposed at about 180° from one and the other. Desirably, the band includes a metal, for example, stainless steel.

### CROSS-REFERENCE TO RELATED APPLICATIONS:

This application claims the benefit of U.S. Provisional Application No. 60/819,422, filed Jul. 7, 2006, the contents of which are incorporated herein by reference.

### FIELD OF THE INVENTION

The present invention relates to devices, methods and systems for delivery and/or repositioning of an implantable stent. More particularly, the present invention relates to a catheter system having coaxial interior and exterior tubes with a stent holder disposed on the interior tube for delivery and/or repositioning of the inplantable stent.

### BACKGROUND

An intraluminal prosthesis is a medical device used in the treatment of diseased bodily lumens. One type of intraluminal prosthesis used in the repair and/or treatment of diseases in various body vessels is a stent. A stent is a generally longitudinal tubular device formed of biocompatible material which is useful to open and support various lumens in the body. For example, stents may be used in the vascular system, urogenital tract, esophageal tract, tracheal/bronchial tubes and bile duct, as well as in a variety of other applications in the body. These devices are implanted within the vessel to open and/or reinforce collapsing or partially occluded sections of the lumen.

Stents generally include an open flexible configuration. This configuration allows the stent to be inserted through curved vessels. Furthermore, this configuration allows the stent to be configured in a radially compressed state for intraluminal catheter implantation. Once properly positioned adjacent the damaged vessel, the stent is radially expanded so as to support and reinforce the vessel. Radial expansion of the stent may be accomplished by inflation of a balloon attached to the catheter or the stent may be of the self-expanding variety which will radially expand once deployed. Tubular shaped structures, which have been used as intraluminal vascular stents, have included helically wound coils which may have undulations or zig-zags therein, slotted stents, ring stents, braided stents and open mesh wire stents, to name a few. Super-elastic materials and metallic shape memory materials have also been used to form stents.

U.S. Pat. Nos. 5,824,041; 6,126,685 and 6,350,278 describe a catheter for use as a delivery device for a radially compressible stent. The catheter has an inner shaft with four rod-shaped stays extending radially from the shaft. The stays are described as being useful for engaging portions of a stent during delivery and/or repositioning of the stent.

U.S. Pat. Nos. 5,733,325; 5,843,167; 5,891,193; 5,902,334; 5,935,161; 5,961,546 and 6,077,297 describe a positioning device for a graft having an exposed terminal anchor within a body lumen. The positioning device has a retention device for engaging the anchor. The retention device includes a central hub and six shafts or spokes extending radially from the hub. The spokes are described as being useful from engaging exposed portions of the anchor. The retention device is described as being mounted on a positioning tube or being an integral part of a disk-shaped stay disposed over the positioning tube.

These stent retention devices, however are high profile devices where the radially extending spokes or rods substantially increase the distance between the inner shaft of a delivery catheter and an outer sheath of the catheter.

Thus, there is a need for an improved stent delivery device. In particular, there is a need for a stent delivery device which can deliver and/or reposition an implantable stent without increasing the overall profile of the device.

### SUMMARY OF THE INVENTION

The invention provides a device for intraluminally delivering a distensible stent. The device includes a first elongate tubular member having a proximal end and a distal end; a band circumferentially disposed over at least a circumferential portion of the first tubular member at the proximal end, the band having at least one projection for releasably engaging a portion of a radially distensible stent; and a second elongate tubular member slidably disposed over the first tubular member and the band. Desirably, the at least one projection is a low-profile, lobate-shaped projection. The band may include two opposed projections, where the two opposed projections may be circumferentially disposed at about 180° from one and the other. Desirably, the band includes a metal, for example, stainless steel.

The first and/or second tubular members may be made from a polymeric material. Useful polymeric materials include polyethylene, polypropylene, polyvinyl chloride, polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters, and copolymers and combinations thereof. The first and/or second tubular member may further include a strand, such as a metallic strand, for reinforcing the tube. The first tube and the second tube may be of the same material or may be different.

In one aspect of the invention, the band may substantially encompass the circumferential portion of the first tubular member. In another aspect of the invention, the band may partially encompass the circumferential portion of the first tubular member. The at least one projection may extend radially outward from the band. Alternatively, or in addition to, the at least one projection may extend longitudinally outward from the band.

In another aspect of the invention, the device is part of a catheter, desirably, a rapid-exchange catheter. The rapid-exchange catheter may include a catheter shaft including the first tubular member and the second tubular member, the first tubular member having a guide wire lumen extending from a proximal guide wire opening disposed distal of the proximal end of the first tubular member to a distal guide wire opening disposed at the distal end of the first tubular member, the first tubular member extending substantially the length of the catheter shaft, the second tubular member having a guide wire opening disposed within the second tubular member distal of the proximal end of the second tubular member, the second tubular member extending substantially the length of the catheter shaft, and the guide wire opening of the second tubular member having a guide wire ramp extending into the proximal guide wire opening of the first tubular member.

The device of this aspect of the invention may further include a radially distensible stent. Desirably, the stent is a braided stent having atraumatic opposed open ends. The stent may further include a liner, a covering, a coating, a graft and combinations thereof.

In yet another aspect of the invention, a delivery system for intraluminally delivering a radially distensible stent is provided. The system may include a radially distensible stent having a proximal and a distal end; and a catheter including a first elongate tubular member having a proximal end and a distal end; a band circumferentially disposed over at least a portion of the first tubular member at the proximal end, the band having at least one projection for releasably engaging a portion of the proximal end of the stent; and a second elongate tubular member slidably disposed over the first tubular member, the band and the stent. Desirably, the at least one projection is a low-profile projection. Useful low-profile projections include, but are not limited to, round projections, roundish projections, semicircular projections, lobate-shaped projections, fin-shaped projections and the like. A band having two opposed projections, desirably low-profile projections, is also useful. Desirably, the two projections are circumferentially disposed at about 180° from one and the other. The band may be a metallic band, a polymeric band and combinations thereof. Useful metals or alloys include, but not limited to, nitinol, stainless steel, cobalt-based alloy such as Elgiloy, platinum, gold, titanium, tantalum, niobium, polymeric materials and combinations thereof. Useful polymeric materials include, but are not limited to, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, fluoroethylene-propylene (FEP), polytetrafluoroethylenes and combinations thereof Heat shrinkable polymers and copolymers are also useful. The stent may further include a liner, a covering, a coating, a graft and combinations thereof.

The first and/or second tubular members in this aspect of the invention may include a polymeric material, such as polyethylene, polypropylene, polyvinyl chloride. polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters, and copolymers and combinations thereof. The tubes may also be reinforced polymeric tubes, for example tubes having polymeric and/or metallic filaments, including braided filaments. The first tube and the second tube may be of the same material or may be different.

The band in this aspect of the invention may substantially encompass the circumferential portion of the first tubular member, or the band may partially encompass the circumferential portion of the first tubular member. The at least one projection may extend radially and/or longitudinally outward from the band.

Desirably, in this aspect of the invention the catheter is a rapid-exchange catheter, which may include a catheter shaft including the first tubular member and the second tubular member, the first tubular member having a guide wire lumen extending from a proximal guide wire opening disposed distal of the proximal end of the first tubular member to a distal guide wire opening disposed at the distal end of the first tubular member, the first tubular member extending substantially the length of the catheter shaft, the second tubular member having a guide wire opening disposed within the second tubular member distal of the proximal end of the second tubular member, the second tubular member extending substantially the length of the catheter shaft; and the guide wire opening of the second tubular member having a guide wire ramp extending into the proximal guide wire opening of the first tubular member.

Desirably, in this aspect of the invention the ends of the stent are atraumatic ends, i.e., ends having not sharp terminating wire ends. Even so, some embodiments of the invention may include free ending wires. The stent may further include a liner, a covering, a coating, a graft and combinations thereof.

In still another aspect of the invention, use or a method for intraluminally delivering a distensible stent is provided. The use or method includes the steps of providing a radially distensible, self-expanding stent having a proximal and a distal end releasably disposed on a catheter; the catheter including a first elongate tubular member having a proximal end and a distal end; a band circumferentially disposed over at least a portion of the first tubular member at the proximal end, the band having at least one projection for releasably engaging a portion of the proximal end of the stent; and a second elongate tubular member slidably disposed over the first tubular member, the band and the stent; wherein the stent is releasably disposed between the tubular members; positioning the catheter within a bodily lumen; slidably retracting the second tubular member from the first tubular member to uncover portions of the stent, whereby the uncovered portions of the stent radially expand against a wall of the bodily lumen; and slidably retracting the second tubular member from the band to release the proximal end of the stent from the band.

In a further aspect of the invention, a use or method for repositioning a radially distensible stent within a bodily lumen is provided. The use or method includes the steps of providing a radially distensible, self-expanding stent having a proximal and a distal end releasably disposed on a catheter; the catheter including a first elongate tubular member having a proximal end and a distal end; a band circumferentially disposed over at least a portion of the first tubular member at the proximal end, the band having at least one projection for releasably engaging a portion of the proximal end of the stent; and a second elongate tubular member slidably disposed over the first tubular member, the band and the stent; wherein the stent is releasably disposed between the tubular members; positioning said catheter within a bodily lumen to a first position; slidably retracting the second tubular member from the first tubular member to uncover only a portion of said stent, whereby the uncovered portion of the stent may radially expand against a wall of the bodily lumen; slidably extending the second tubular member over the first tubular member to recover the a portion of the stent; and repositioning the catheter within a bodily lumen from the first position to a second position. The method may further include the step of slidably retracting the second tubular member from the band to release the proximal end of the stent from the band at said second position.

### BRIEF DESCRIPTION OF THE DRAWINGS

FIG. 1 is a perspective view of a two coaxially slidable tubes useful for delivering and/or repositioning an implantable stent according to one embodiment of the invention.

FIG. 2 is a planar view of the tubes of FIG. 1 further illustrating the ability of the tubes to be slid over one and the other.

FIG. 3 is a cross-sectional view of inner tube of FIG. 2 taken along the 3-3 axis.

FIG. 4 is a cross-sectional view of the outer tube of FIG. 2 taken along the 4-4 axis.

FIG. 5 is a planar view of the inner tube of FIG. 2 further depicting a stent holder disposed on one end of the tube.

FIG. 6 is a planar view of the tube of FIG. 5 further depicting a stent disposed over the tube.

FIG. 7A is a perspective view of a stent holder according to one embodiment of the invention.

FIG. 7B is a perspective view of a stent holder of FIG. 7A engaging a portion of a proximal end of a stent according to one embodiment of the invention.

FIG. 8 is a cross-sectional view of the stent holder of FIG. 7 taken along the 8-8 axis.

FIG. 9 is a perspective view of another embodiment of a stent holder according to one embodiment of the invention.

FIG. 10 is a cross-sectional view of the stent holder of FIG. 9 taken along the 10-10 axis.

FIG. 11 is a perspective view of still another embodiment of a stent holder of one embodiment of the invention.

FIG. 12 is a perspective view of yet another embodiment of a stent holder of one embodiment of the invention.

FIG. 13 is a planar depiction of partial deployment of a stent within a body lumen according to one embodiment of the invention.

FIG. 14 is a planar view of a rapid exchange stent delivery catheter system according to one embodiment of the invention.

FIG. 15 is a planar view of a distal portion of the rapid exchange stent delivery catheter system of FIG. 14, shown in a deployment state.

FIG. 16 is a longitudinal view of a wire stent of one embodiment of the invention.

FIG. 17 is a longitudinal view of an atraumatic braided stent of one embodiment of the invention.

FIG. 18 is a longitudinal view of a zig-zag stent of one embodiment of the invention.

FIG. 19 is a longitudinal view of an alternate zig-zag stent of one embodiment of the invention.

FIG. 20 is a perspective view of slotted stent of one embodiment of the invention.

FIG. 21 is a perspective view of a helical coil stent formed of a single wound wire according to one embodiment of the invention.

FIG. 22 is a perspective view of a stent having an elongate pre-helically coiled configuration according to one embodiment of the invention.

FIG. 23 is a schematic depiction for forming the stent holder of FIG. 9 from a flat substrate.

FIGS. 24A-24B are schematic depictions for forming the stent holder of FIG. 7A from a flat substrate or substrates.

### DETAILED DESCRIPTION OF THE PREFERRED EMBODIMENT

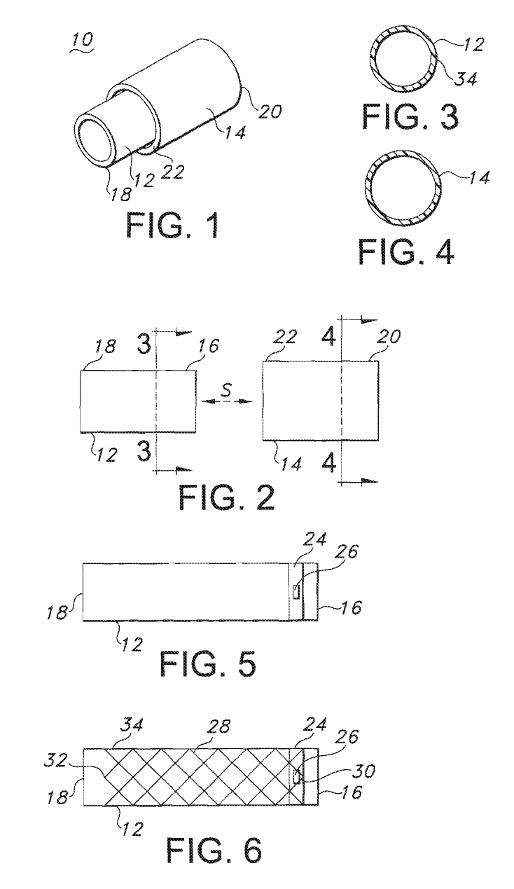

FIG. 1 is a perspective view of an endoprosthesis or stent delivery device 10 of one embodiment of the invention. The delivery device includes coaxially disposed interior tube 12 and exterior tube 14. As depicted in FIG. 2, the interior tube 12 and the exterior tube 14 are slidingly disposed to one and the other, as indicated by vector “S”. As such, exterior tube 14 may be slid over the interior tube 12 and/or the interior tube 12 may be slid within the exterior tube 14.

As depicted in FIGS. 1 and 2, the interior tube 12 has a proximal end 16 and an opposed distal end 18; and the exterior tube 14 has a proximal end 20 and a distal end 22. It should be noted that references herein to the term “distal” are to a direction away from an operator of the subject invention, while references to the term “proximal” are to a direction towards the operator of the subject invention. As depicted in FIG. 3, which is a cross-section view of the interior tube 12 of FIG. 2 taken along the 3-3 axis, the interior tube 12 is a hollow tube. As depicted in FIG. 4, which is a cross-section view of the exterior tube 14 of FIG. 2 taken along the 4-4 axis, the exterior tube 14 is a hollow tube.

The interior tube 12 and/or the exterior tube 14 may be constructed of any suitable biocompatible materials, such as, but not limited to, polymeric polymers and materials, including fillers such as metals, carbon fibers, glass fibers or ceramics, and combinations thereof. Useful, but non-limiting, polymeric materials include polyethylene, polypropylene, polyvinyl chloride, polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters, and copolymers and combinations thereof. Further, the interior tube 12 and/or the exterior tube 14 may be reinforced to provide greater strength while minimizing overall tube profile. For example, the interior tube 12 and/or the exterior tube 14 may have a reinforcing material, for example a polymeric, metallic or ceramic strand or tape, encased within the tube or otherwise disposed on or within the tube. The reinforcing strand or tape may be braided, woven, wound, and the like to form a reinforcing member for the tube.

As depicted in FIG. 5, a stent holder 24 may be disposed at or near the proximal end 16 of the interior tube 12. The stent holder 24 may include a projection 26, which is useful for holding or securing a stent during use of the device 10. As depicted in FIG. 6 a stent 28 is disposed over the outer surface 34 of the interior tube 12. The stent 28 is a hollow tubular device with an open lattice wall structure having a proximal end 30 and an opposed distal end 32. As illustrated in FIG. 6, the projection 26 of the stent holder 24 securably holds or retains a portion of the proximal end 30 of the stent 28.

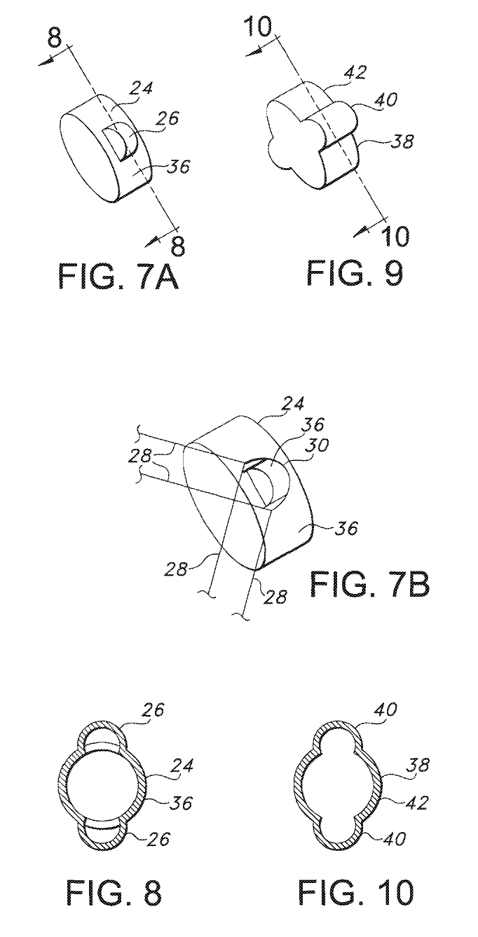

FIG. 7A is a perspective view of the stent holder 24 of FIG. 6. The stent holder 24 may include a circumferential band 36 from which the projection 26 outwardly extends in a radial direction. FIG. 8 is a cross-section view of the stent holder 24 of FIG. 7A taken along the 8-8 axis. As depicted in FIG. 8 the stent holder 24 is hollow tubular device. Desirably, the stent holder 26 may include a pair of opposed projections 26. Desirably, the projections 26 are opposed from one and the other or in other words are disposed at about 180° from one and the other. As depicted in FIG. 8, the projection 26 is a low-profile projection. Useful low-profile projections include, but are not limited to, round projections, roundish projections, semicircular projections, lobate-shaped projections, fin-shaped projections and the like. As depicted in FIG. 7B, such a shaped projection 26 is useful for engaging the proximal end 30 of the stent 28. While the proximal end 30 of the stent 28 is depicted as a closed-end wire loop in FIG. 7B, the invention is not so limited and as described below other stent configuration may suitably be used. Moreover, as contrasted to the attenuated rod or pin shaped projections of the prior art, rounded and blunt shape of the projection 26 of one embodiment of the invention offers lower profile, i.e., reduced height, while offering greater stent-projection contacting areas to grip the stent 28 during deployment, reconstraining and/or repositioning of the stent 28 during intraluminal delivery. Further, as depicted in FIG. 8, the projection 26 may also be a hollow member. Projection 26 may be fully or partially elastic to adapt to compress in between the inner and outer tubes 12, 14 or to better releasably grasp and/or hold the stent 28. Projection 26 may also be a coated projection, such as a metal or stainless steel coated with an elastic polymer. Further, the projection 26 may include a material, such as a polymeric material, having a degree of tackiness to better releasably grasp and/or hold the stent 28.

Desirably, the band 36 of the stent holder 24 is as thin as possible to reduce the overall profile of the holder 24. The holder 24 may be constructed from any biocompatible metal, desirably stainless steel, or polymeric material.

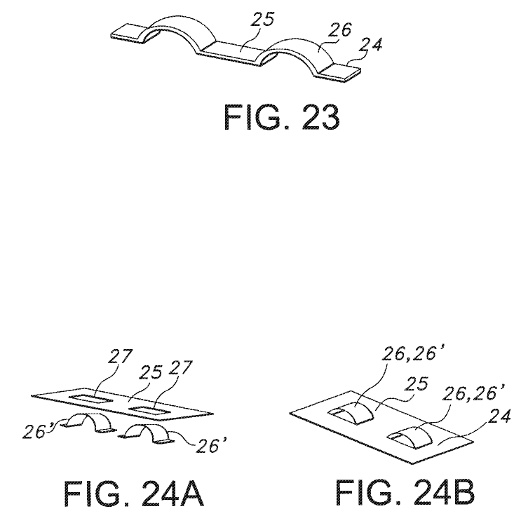

The holder 24 may be manufactured by any suitable technique, such as, but not limited to, electrical discharge machining, metal injection molding. Further, the holder 24 may be made by using metal stamping technology. For example as depicted in FIG. 23, a flat piece of metal 25, for example stainless steel, could be stamped to shape the projections 24. The shaped metallic band could then be shaped around the interior tube 12 and glued, crimped or swaged in place. Alternatively as depicted in FIGS. 24A-B, slots 27 may be cut into a flat piece 25 of metal and the shaped projections 26′ may be inserted into the slots, followed by shaping the assembly around the interior tube 12 and fastening the shaped band thereon.

The invention is not limited to the shape of the stent holder 24 as depicted in FIGS. 7A-8, and other low profile stent holder configurations may suitably be used. For example, as depicted in FIGS. 9-10, a stent holder 38 may include a pair of opposed projections 40 extending radially outward from a circular band 42, where the longitudinal length of the band 42 and the longitudinal length of the projections 40 are substantially similar.

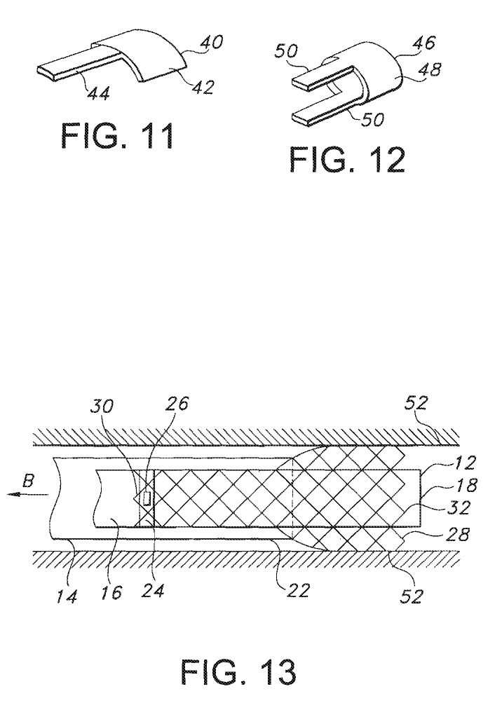

Moreover, the invention is not limited to radially outwardly projecting stent holders 24, 38 as depicted in FIGS. 7A-8, and other low profile stent holder projection configurations may suitably be used. For example, as depicted in FIG. 11, stent holder 40 includes a band 42 and a tab or projection 44. Band 42 is depicted as being a partial circular member. Desirably, the band 42 encompasses about half or less of the circumference of the exterior surface 34 at the proximal end 16 of the interior tube 12. The tab 44 extends substantially longitudinally from the band 42. The tab 44 may have a raised portion (not shown) to facilitate the gripping of the stent 28. As depicted in FIG. 12, stent holder 46 may include a substantially circular band 48 and two tabs or projections 50 extending substantially longitudinally from the band 48. The tab 50 may have a raised portion (not shown) for gripping the stent 28. The tabs 44 and 50, however, are useful for gripping the stent 28 without having raised portions.

FIG. 13 depicts partial deployment of the stent 28 with the device 10 of one embodiment of the invention. After the device 10 is placed within a body lumen 52, the exterior tube 14 may be retracted or slid away from the interior tube 12. As exterior tube 14 retracts, the exposed distal portion 32 of the stent 28 expands against the walls of the body lumen 52. When distal end 22 of exterior tube 14 is retracted past the proximal portion 16 of the interior tube 12 having the stent holder 24 disposed thereon, the stent 28 may be fully deployed with the body lumen 52. The device 10 may be retracted from the body lumen 52, leaving the deployed stent 28 within the body lumen 52. Prior to full deployment of the stent 28, i.e., prior to retraction of the distal potion 22 of the exterior tube 14 past the stent holder 24 disposed on the proximal end 16 of the interior tube, the stent 28 may be repositioned within the body lumen. The exterior tube 14 may be reslid over the interior tube 12 to reconstrain the stent 28 therebetween. The device 10 may then be repositioned within the body lumen 52, followed by redeployment of the stent 28.

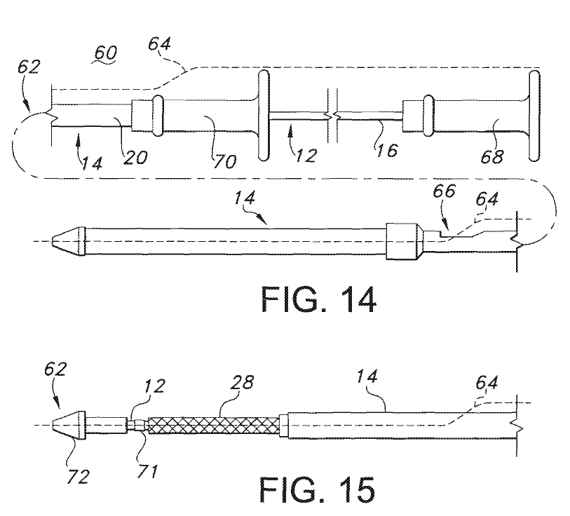

In another aspect of the invention a plan view of a rapid exchange stent delivery catheter system 60 is illustrated in FIGS. 14-15. The rapid exchange stent delivery catheter system 60 includes a rapid exchange catheter 62 which is advanced over a guide wire 64 (shown in phantom) to deliver and deploy the self-expanding stent 28 in a bodily lumen. The guidewire 64 may be any guidewire as is known in the art. Guidewire 64 is typically an elongated, relatively rigid, but typically flexible, cylindrical member. Guidewire 64 may be constructed of any material, but is preferably constructed of metal, such as stainless steel, gold, platinum, and metal alloys such as cobalt-based alloys or titanium alloys, for example, nickel-titanium shape memory alloys (i.e., nitinol), titanium-aluminum-vanadium alloys and titanium-zirconium-niobium alloys. Moreover, guidewire 64 may have a constant stiffness or flexibility along the entire length thereof, or may have portions of varying stiffness and flexibility, such as an area of increased flexibility at guidewire tip 64. Guidewire 64 may further include a coating along a portion or the entire length thereof such as a lubricious or frictionless coating material. Guidewire 64 may further be provided with a radiopaque portion, for example in the form of a radiopaque coating on a portion of the guidewire, or by constructing a portion of the guidewire out of a radiopaque material.

The rapid exchange stent delivery catheter system 62 is suitable for intraluminal applications, including, but not limited to, biliary applications and intravascular applications. In biliary applications, the rapid exchange stent delivery catheter system 62 may be sized to fit within an endoscope (not shown) and to navigate to the desired site in the biliary tract. In vascular applications, the rapid exchange stent delivery catheter system 62 may be sized to fit within an introducer sheath (not shown) and/or a guide catheter (not shown) to navigate to the desired vascular site.

The rapid exchange stent delivery catheter 62 includes the inner tubular member 12 slidably disposed in the outer tubular member 14. The outer tubular member 14 includes a lumen (not visible) extending therethrough to slidably accommodate the inner tubular member 12. The inner tubular member 12 includes a guide wire lumen extending through a distal portion thereof to accommodate the guide wire 64.

To provide rapid exchange capability for the rapid exchange stent delivery catheter 62, the guide wire 64 exits through a guide wire opening 66 in the outer tubular member 14. The guide wire 64 extends through a relatively short guide wire lumen and enters through a distal guide wire opening in the inner tubular member 12. In practice, the device 62 may be inserted over the guide wire 66 from the tip end first.

A proximal handle 68 is connected to a proximal portion 16 of the inner tubular member 12. Similarly, a distal handle 70 is connected to a proximal portion 20 of the outer tubular member 14. The distal handle 70 may be longitudinally displaced relative to the proximal handle 68 to selectively expose or cover the stent 28. In FIG. 14, the distal handle 70 has been longitudinally displaced in the distal direction relative to proximal handle 68 such that the outer tubular member 14 covers the stent 28. In FIG. 15, the distal handle 70 has been longitudinally displaced in the proximal direction relative to proximal handle 68 to retract the outer tubular member 14 relative to the inner tubular member 12 to expose and deploy the stent 28.

A distal head 72 may be connected to the distal end of the distal inner portion of the inner tubular ember 12 to limit, if desired, distal displacement of the outer tubular member 14. Radiopaque marker bands, for example marker 71, may be on the catheter 62 to facilitate placement of the device 62 during intraluminal delivery. The markers may include any useful radiopaque material or materials including any metal or plastics being radiopaque or capable of being impregnated with radiopaque materials. Useful radiopaque materials include, but are not limited to gold, barium sulfate, ferritic particles, platinum, platinum-tungsten, palladium, platinum-iridium, rhodium, tantalum or combinations thereof. The stent holder 24 itself may also be made of or include radiopaque materials.

Additional details of suitable catheters, including rapid exchange catheters and systems, may be found in U.S. Pat. No. 6,723,071, the contents of which are incorporated herein by reference.

The stent 28 may be made from any suitable implantable material, including without limitation nitinol, stainless steel, cobalt-based alloy such as Elgiloy®, platinum, gold, titanium, tantalum, niobium, polymeric materials and combinations thereof. Useful and nonlimiting examples of polymeric stent materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L,-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like. Further, the stent 28, or portions of the stent 28, may have a composite construction, such as described found in U.S. Patent Application Publication 2002/0035396 A1, the contents of which is incorporated herein by reference. For example, the stent 28 may have an inner core of tantalum gold, platinum, iridium or combination of thereof and an outer member or layer of nitinol to provide a composite wire for improved radiocapicity or visibility. Preferably, the stent 28 is made from nitinol.

Also, the stent 28 may be treated with any known or useful bioactive agent or drug including without limitation the following: anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

The stent may be coated with a polymeric material. For example, the stent wires may be partially or fully covered with a biologically active material which is elutably disposed with the polymeric material. Further, the polymeric coating may extend over or through the interstitial spaces between the stent wires so as to provide a hollow tubular liner or cover over the interior or the exterior surface of the stent, thereby providing a stent-graft device. The polymeric material may be selected from the croup consisting of polyester, polypropylene, polyethylene, polyurethane, polynaphthalene, polytetrafluoroethylene, expanded polytetrafluoroethylene, silicone, and combinations thereof. The covering may be in the form of a tubular structure. The silicone covering may be suitably formed by dip coating the stent. Details of such dip coating may be found in U.S. Pat. No. 5,875,448, the content of which is incorporated herein by reference. The present invention is not limited to forming the silicone film by dip coating, and other techniques, such as spraying, may suitably be used. After applying the silicone coating or film to the stent, the silicone may be cured. Desirably, the curing is low temperature curing, for example from about room temperature to about 90° C. for a short period of time, for example from about 10 minutes or more to about 16 hours. The cured silicone covering may also be sterilized by electronic beam radiation, gamma radiation ethylene oxide treatment and the like. Further details of the curing and/or sterilization techniques may be found in U.S. Pat. No. 6,099,562, the content of which is incorporated herein by reference. Argon plasma treatment of the cured silicone may also be used. Argon plasma treatment of the cured silicone modifies the surface to the cured silicone to, among other things, make the surface less sticky. The invention, however, is not limited to stent-graft devices having polymeric coatings. The graft portion may suitably be formed from polymeric films, polymeric tapes, polymeric tubes, polymeric sheets and textile materials. Textile material may be wovens, knitted, braided and/or filament wound to provide a suitable graft. Various biocompatible polymeric materials may be used as textile materials to form the textile structures, including polyethylene terephthalate (PET), naphthalene dicarboxylate derivatives such as polyethylene naphthalate, polybutylene naphthalate, polytrimethylene naphthalate, trimethylenediol naphthalate, ePTFE, natural silk, polyethylene and polypropylene, among others. Moreover, textile materials and stent materials may be co-formed, for example co-braided, to form a stent-graft device.

Various stent types and stent constructions may be employed in the invention. Among the various stents useful include, without limitation, self-expanding stents and balloon expandable extents. The stents may be capable of radially contracting, as well and in this sense can best be described as radially distensible or deformable. Self-expanding stents include those that have a spring-like action which causes the stent to radially expand, or stents which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other biocompatible metals, as well as polymeric stents, including biodegradable and bioabsorbable stents. The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like. Examples of various stent configurations are shown in U.S. Pat. No. 4,503,569 to Dotter; U.S. Pat. No. 4,733,665 to Palmaz; U.S. Pat. No. 4,856,561 to Hillstead; U.S. Pat. No. 4,580,568 to Gianturco; U.S. Pat. No. 4,732,152 to Wallsten, U.S. Pat. No. 4,886,062 to Wiktor, and U.S. Pat. No. 5,876,448 to Thompson, all of whose contents are incorporated herein by reference.

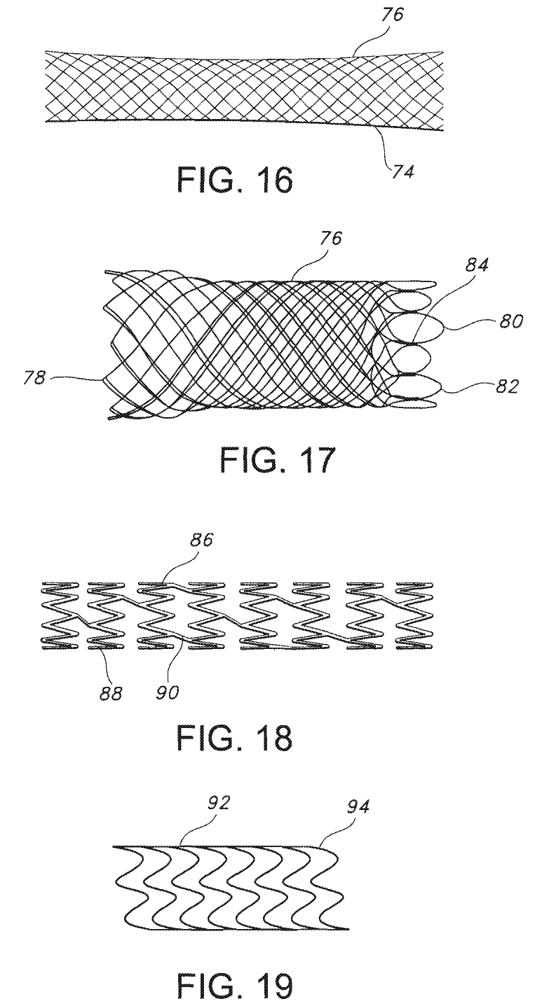

As described above, various stent types and stent constructions may be employed in the invention as the stent 28. Non-limiting examples of suitable stent geometries for stent 28 are illustrated in FIGS. 16-22. As shown in FIG. 16, wire stent 74 is a hollow tubular structure formed from wire strand 76 or multiple wire strands. Wire stent 74 may be formed by, for example, braiding or spinning wire strand(s) 76 over a mandrel (not shown). Wire stent 74 is capable of being radially compressed and longitudinally extended for implantation into a bodily lumen. The degree of elongation depends upon the structure and materials of the wire stent 74 and can be quite varied, for example, about 5% to about 200% of the length of wire stent 74. The diameter of wire stent 74 may also become several times smaller as it elongates. Unitary stent structures may be obtained by braiding and/or filament winding stent wires to obtain complex stent geometries, including complex stent geometries, including complex bifurcated stents. Alternatively, stent components of different sizes and/or geometries may be mechanically secured by welding or suturing. Additional details of wire stents of complex geometry are described in U.S. Pat. Nos. 6,325,822 and 6,585,758, the contents of which are incorporated herein by reference.

As depicted in FIG. 17, braided stent 76 is desirably an atraumatic stent having no sharp terminating members at one or both of the opposed open ends 78, 80. The elongate stent wires terminating at open end 80 are mated to form closed loops 82 and adjacently mated wires are secured to one and the other by mechanical means, such as welds 84. The positioning of adjacently mated wires to form closed-loop end designs is further described in U.S. Patent Application Publication Nos. 2005/0049682 A1 and 2006/0116752 A1, the contents of which are incorporated herein by reference. Desirably, the elongate wires terminating at open end 80 are in a cathedral type arch or loop configuration. Further details of the cathedral type of arch or closed-loop configuration may be found in U.S. Patent Application Publication No. 2005/0256563 A1, the contents of which are incorporated herein by reference. The stent wires at the opposed open end 78 may also be free of any sharp terminating points by, for example, commencing braiding of the wires under tension over a pin (not shown) so that the wire ends terminate just at the end 80, where the wire ends may be looped and welded thereat.

A zig-zag wire stent 86 may also be useful as stent 28. Wire strand 88 may be arranged in what can be described as a multiple of “Z” or “zig-zag” patterns to form a hollow tubular stent. The different zig-zag patterns may optionally be connected by connecting member 90. Further, zig-zag wire stent 86 is not limited to a series of concentric loops as depicted in FIG. 18, but may be suitably formed by helically winding of the “zig-zag” pattern over a mandrel (not shown). For example, as depicted in FIG. 19, zig-zag stent 92 is formed by helically winding at least one stent wire 94 with no interconnections between the helically wound undulating portions. The wire ends (not shown) may be looped and welded to provide no sharp wire ends at the ends of the stent.

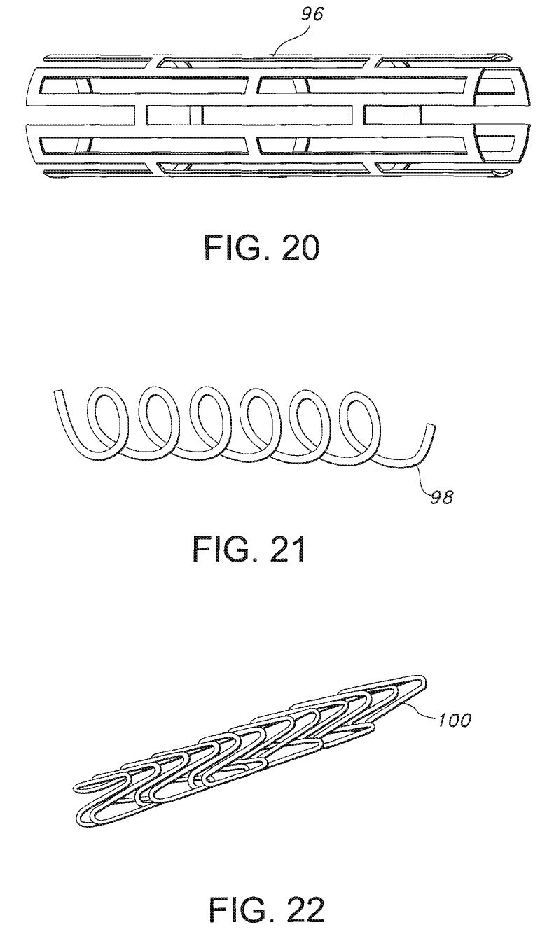

A slotted stent 96 may also be useful as stent 28. As depicted in FIG. 20, slotted stent 96 is suitably configured for implantation into a bodily lumen (not shown). Upon locating the slotted stent 96 at the desired bodily site, slotted stent 96 is radially expanded and longitudinally contracted for securement at the desired site.

Other useful stents capable of radial expansion are depicted in FIGS. 21 and 22. As depicted in FIG. 21, stent 98 is a helical coil which is capable of achieving a radially expanded state (not shown). Stent 100, as depicted in FIG. 2, has an elongate pre-helically coiled configuration as shown by the waves of non-overlapping undulating windings. These helically coiled or pre-helically stents, commonly referred to as nested stents, are also useful with the practice of one embodiment of the invention.

In another aspect of the invention, uses of the devices or systems or methods for intraluminally delivering a distensible stent are provided. The use or method may include the steps of providing a radially distensible, self-expanding stent having a proximal and a distal end releasably disposed on a catheter; the catheter including a first elongate tubular member having a proximal end and a distal end; a band circumferentially disposed over at least a portion of the first tubular member at the proximal end, the band having at least one projection for releasably engaging a portion of the proximal end of the stent; and a second elongate tubular member slidably disposed over the first tubular member, the band and the stent; wherein the stent is releasably disposed between the tubular members; positioning the catheter within a bodily lumen; slidably retracting the second tubular member from the first tubular member to uncover portions of the stent, whereby the uncovered portions of the stent radially expand against a wall of the bodily lumen; and slidably retracting the second tubular member from the band to release the proximal end of the stent from the band. The stent may further include a liner, a covering, a coating, a graft and combinations thereof.

In another aspect of the invention, use or a method for repositioning a radially distensible stent within a bodily lumen is provided. The method includes the steps of providing a radially distensible, self-expanding stent having a proximal and a distal end releasably disposed on a catheter; the catheter including a first elongate tubular member having a proximal end and a distal end; a band circumferentially disposed over at least a portion of the first tubular member at the proximal end, the band having at least one projection for releasably engaging a portion of the proximal end of the stent; and a second elongate tubular member slidably disposed over the first tubular member, the band and the stent; wherein the stent is releasably disposed between the tubular members, positioning said catheter within a bodily lumen to a first position; slidably retracting the second tubular member from the first tubular member to uncover only a portion of said stent, whereby the uncovered portion of the stent may radially expand against a wall of the bodily lumen; slidably extending the second tubular member over the first tubular member to recover the a portion of the stent; and repositioning the catheter within a bodily lumen from the first position to a second position. The method may further include the step of slidably retracting the second tubular member from the band to release the proximal end of the stent from the band at said second position. The stent may further include a liner, a covering, a coating, a graft and combinations thereof.

In another aspect of the invention, a catheter for intraluminally delivering a distensible stent is provided. The catheter includes a first elongate tubular member having a proximal end and a distal end; a band circumferentially disposed over at least a portion of the first tubular member at the proximal end, the band having at least one projection for releasably engaging a portion of a radially distensible stent; and a second elongate tubular member slidably disposed over the first tubular member and the band. The catheter may be a rapid-exchange catheter. The stent may further include a liner, a covering, a coating, a graft and combinations thereof.

In another aspect of the invention, a rapid-exchange catheter for intraluminally delivering a distensible stent includes a first elongate tubular member having a proximal end and a distal end; a band circumferentially disposed over at least a portion of the first tubular member at the proximal end, the band having at least one projection for releasably engaging a portion of a radially distensible stent; and a second elongate tubular member slidably disposed over the first tubular member and the band; wherein the rapid-exchange catheter may further include a catheter shaft including the first tubular member and the second tubular member, the first tubular member having a guide wire lumen extending from a proximal guide wire opening disposed distal of the proximal end of the first tubular member to a distal guide wire opening disposed at the distal end of the first tubular member, the first tubular member extending substantially the length of the catheter shaft, the second tubular member having a guide wire opening disposed within the second tubular member distal of the proximal end of the second tubular member, the second tubular member extending substantially the length of the catheter shaft; and the guide wire opening of the second tubular member having a guide wire ramp extending into the proximal guide wire opening of the first tubular member. The stent may further include a liner, a covering, a coating, a graft and combinations thereof.

The invention being thus described, it will now be evident to those skilled in the art that the same may be varied in many ways. Such variations are not to be regarded as a departure from the spirit and scope of the invention and all such modifications are intended to be included within the scope of the following claims. Further, any of the embodiments or aspects of the invention as described in the claims or in the specification may be used with one and another without limitation.

What is claimed is:

1. A device for intraluminally delivering a distensible stent comprising:
a first elongate tubular member having a proximal end and a distal end;
a band circumferentially disposed over at least a portion of said first tubular member at said proximal end, said band having at least one projection for releasably engaging a portion of a radially distensible stent; and
a second elongate tubular member slidably disposed over said first tubular member and said band.

2. The device of claim 1, wherein said at least one projection is a low-profile, roundish-shaped projection.

3. The device of claim 1, wherein said band has two opposed projections.

4. The device of claim 3, wherein said two projections are circumferentially disposed at about 180° from one and the other.

5. The device of claim 1, wherein said band comprises a metal.

6. The device of claim 5, wherein said metal is stainless steel.

7. The device of claim 1, wherein said first tubular member comprises a polymeric material.

8. The device of claim 7, wherein said polymeric material is selected from the group consisting of polyethylene, polypropylene, polyvinyl chloride, polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters, and copolymers and combinations thereof.

9. The device of claim 7, wherein said first tubular member further comprises a metallic strand for reinforcing said tube.

10. The device of claim 1, wherein said second tubular member comprises a polymeric material.

11. The device of claim 10, wherein said polymeric material is selected from the group consisting of polyethylene, polypropylene, polyvinyl chloride, polytetrafluoroethylene, fluorinated ethylene propylene copolymer, polyyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters and copolymers and combinations thereof.

12. The device of claim 10, wherein said second tubular member further comprises a metallic strand for reinforcing said tube.

13. The device of claim 1, wherein said band substantially circumferentially encompasses said portion of said first tubular member.

14. The device of claim 1, wherein said band partially circumferentially encompasses said portion of said first tubular member.

15. The device of claim 1, wherein said at least one projection extends radially outward from said band.

16. The device of claim 1, wherein said at least one projection extends longitudinally outward from said band.

17. The device of claim 1, wherein said device is a catheter.

18. The device of claim 17, wherein said catheter is a rapid-exchange catheter.

19. The device of claim 18, wherein said rapid-exchange catheter comprises:
a catheter shaft including said first tubular member and said second tubular member, said first tubular member having a guide wire lumen extending from a proximal guide wire opening disposed distal of said proximal end of said first tubular member to a distal guide wire opening disposed at said distal end of said first tubular member, the first tubular member extending substantially the length of said catheter shaft, said second tubular member having a guide wire opening disposed within said second tubular member distal of said proximal end of said second tubular member, said second tubular member extending substantially the length of said catheter shaft; and said guide wire opening of said second tubular member having a guide wire ramp extending into the proximal guide wire opening of said first tubular member.

20. The device of claim 1, further comprising a radially distensible stent.

21. The device of claim 20, wherein said stent is a braided stent having atraumatic opposed open ends.

22. A delivery system for intraluminally delivering a radially distensible stent comprising:
a radially distensible stent having proximal and distal ends; and
a catheter comprising:
a first elongate tubular member having a proximal end and a distal end;
a band circumferentially disposed over at least a portion of said first tubular member at said proximal end, said band having at least one projection for releasably engaging a portion of said proximal end of said stent; and
a second elongate tubular member slidably disposed over said first tubular member, said band and said stent.

23. The system of claim 22, wherein said at least one projection is a low-profile, roundish-shaped projection.

24. The system of claim 22, wherein said band has two opposed projections.

25. The system of claim 24, wherein said two projections are circumferentially disposed at about 180° from one and the other.

26. The system of claim 22, wherein said band comprises a metal.

27. The system of claim 26, wherein said metal is stainless steel.

28. The system of claim 22, wherein said first tubular member comprises a polymeric material selected from the group consisting of polyethylene, polypropylene, polyvinyl chloride, polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters, and copolymers and combinations thereof.

29. The system of claim 22, wherein said second tubular member comprises a polymeric material selected from the group consisting of polyethylene, polypropylene, polyvinyl chloride, polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, fully or partially halogenated polyethers, polyamide/polyether polyesters, and copolymers and combinations thereof.

30. The system of claim 22, wherein said band substantially circumferentially encompasses said portion of said first tubular member.

31. The system of claim 22, wherein said band partially circumferentially encompasses said portion of said first tubular member.

32. The system of claim 22, wherein said at least one projection extends radially outward from said band.

33. The system of claim 22, wherein said at least one projection extends longitudinally outward from said band.

34. The system of claim 22, wherein said catheter is a rapid-exchange catheter further comprising:
a catheter shaft including said first tubular member and said second tubular member, said first tubular member having a guide wire lumen extending from a proximal guide wire opening disposed distal of said proximal end of said first tubular member to a distal guide wire opening disposed at said distal end of said first tubular member, the first tubular member extending substantially the length of said catheter shaft, said second tubular member having a guide wire opening disposed within said second tubular member distal of said proximal end of said second tubular member, said second tubular member extending substantially the length of said catheter shaft; and said guide wire opening of said second tubular member having a guide wire ramp extending into the proximal guide wire opening of said first tubular member.

35. The system of claim 22, wherein said ends of said stent are atraumatic ends.

36. A, method for intraluminally delivering a distensible stent comprising:
providing a radially distensible, self-expanding stent having a proximal and a distal end releasably disposed on a catheter; said catheter comprising:
a first elongate tubular member having a proximal end and a distal end;
a band circumferentially disposed over at least a portion of said first tubular member at said proximal end, said band having at least one projection for releasably engaging a portion of said proximal end of said stent; and
a second elongate tubular member slidably disposed over said first tubular member, said band and said stent:
wherein said stent is releasably disposed between said tubular members;
positioning said catheter within a bodily lumen;
slidably retracting said second tubular member from said first tubular member to uncover portions of said stent, whereby the uncovered portions of said stent radially expand against a wall of the bodily lumen; and
slidably retracting said second tubular member from said band to release said proximal end of said stent from said band.

37. A method for repositioning a radially distensible stent within a bodily lumen comprising:
providing a radially distensible, self-expanding stent having a proximal and a distal end releasably disposed on a catheter; said catheter comprising:
a first elongate tubular member having a proximal end and a distal end;
a band circumferentially disposed over at least a portion of said first tubular member at said proximal end, said band having at least one projection for releasably engaging a portion of said proximal end of said stent; and
a second elongate tubular member slidably disposed over said first tubular member, said band and said stent;
wherein said stent is releasably disposed between said tubular members;
positioning said catheter within a bodily lumen to a first position;
slidably retracting said second tubular member from said first tubular member to uncover only a portion of said stent, whereby the uncovered portion of said stent may radially expand against a wall of the bodily lumen;
slidably extending said second tubular member over said first tubular member to recover said a portion of said stent; and
repositioning said catheter within a bodily lumen from said first position to a second position.

38. The method of claim 37, further comprising the step of
slidably retracting said second tubular member from said band to release said proximal end of said stent from said band at said second position.

39. The device of claim 20, wherein said stent further comprises a liner, a covering, a coating, a graft and combinations thereof.

40. The system of claim 22, wherein said stent further comprises a liner, a covering, a coating, a graft and combinations thereof.

41. The method of claim 36, wherein said stent further comprises a liner, a covering, a coating, a graft and combinations thereof.

42. The method of claim 37, wherein said stent further comprises a liner, a covering, a coating, a graft and combinations thereof.

43. A catheter for intraluminally delivering a distensible stent comprising:
a first elongate tubular member having a proximal end and a distal end;
a band circumferentially disposed over at least a portion of said first tubular member at said proximal end, said band having at least one projection for releasably engaging a portion of a radially distensible stent; and
a second elongate tubular member slidably disposed over said first tubular member and said band.

44. A rapid-exchange catheter for intraluminally delivering a distensible stent comprising:
a first elongate tubular member having a proximal end and a distal end;
a band circumferentially disposed over at least a portion of said first tubular member at said proximal end, said band having at least one projection for releasably engaging a portion of a radially distensible stent; and
a second elongate tubular member slidably disposed over said first tubular member and said band.

45. A rapid-exchange catheter for intraluminally delivering a distensible stent comprising:
a first elongate tubular member having a proximal end and a distal end;
a band circumferentially disposed over at least a portion of said first tubular member at said proximal end, said band having at least one projection for releasably engaging a portion of a radially distensible stent; and
a second elongate tubular member slidably disposed over said first tubular member and said band.
wherein said rapid-exchange catheter further comprises a catheter shaft including said first tubular member and said second tubular member, said first tubular member having a guide wire lumen extending from a proximal guide wire opening disposed distal of said proximal end of said first tubular member to a distal guide wire opening disposed at said distal end of said first tubular member, the first tubular member extending substantially the length of said catheter shaft, said second tubular member having a guide wire opening disposed within said second tubular member distal of said proximal end of said second tubular member, said second tubular member extending substantially the length of said catheter shaft; and said guide wire opening of said second tubular member having a guide wire ramp extending into the proximal guide wire opening of said first tubular member.
